(19) United States
(12) Patent Application Publication
(10) Pub. No.: US 2018/0116495 A1
(43) Pub. Date: May 3, 2018

(54) HIGH-PRESSURE ENDOSCOPE CLEANING DEVICE

(71) Applicant: Cook Medical Technologies LLC, Bloomington, IN (US)

(72) Inventors: John A. Karpiel, JR., Winston-Salem, NC (US); Gregory J. Skerven, Kernersville, NC (US)

(73) Assignee: Cook Medical Technologies LLC, Bloomington, IN (US)

(21) Appl. No.: 15/798,903

(22) Filed: Oct. 31, 2017

Related U.S. Application Data
(60) Provisional application No. 62/415,317, filed on Oct. 31, 2016.

(51) Int. Cl. A61B 1/12; B08B 9/032; B08B 9/023; B05B 1/02; B05B 13/04; B05B 13/06; B05B 15/06

(52) U.S. Cl. CPC A61B 1/125; B08B 9/0321; B08B 9/023; B05B 15/066; B05B 13/04; B05B 13/0627; B05B 1/02

(57) ABSTRACT

In one aspect, the present disclosure provides a device for cleaning an endoscope. The device may include an elongate housing with a cavity configured to receive at least a portion of a medical device. The device may further include an inlet in fluid communication with the cavity, the inlet configured to connect to a source for providing a cleaning fluid at a pressure to the cavity through the inlet, the pressure being 100 psi or more. The device may further include an outlet in fluid communication with the cavity, the outlet configured to remove the cleaning fluid from the cavity.

### CROSS-REFERENCE TO RELATED APPLICATIONS

This application claims the benefit of U.S. Provisional Application Ser. No. 62/415,317, filed Oct. 31, 2016, which is hereby incorporated by reference in its entirety.

### BACKGROUND

Endoscopic devices and procedures may be used to diagnose, monitor and treat various conditions by close examination of the internal organs. By way of background, a conventional endoscope generally is an instrument having a device for visualizing the interior of an internal region of a body and a lumen for inserting one or more treatment devices therethrough. A wide range of applications have been developed for the general field of endoscopes including by way of example the following: an arthroscope, an angioscope, a bronchoscope, a choledochoscope, a colonoscope, a cytoscope, a duodenoscope, a enteroscope, a esophagogastro-duodenoscope (gastroscope), a laparoscope, a laryngoscope, a nasopharyngo-neproscope, a sigmoidoscope, a thoracoscope, and an utererscope (individually and collectively, “endoscope”).

During use in surgical procedures, an endoscope typically becomes soiled with biological and other materials from a patient body (e.g., biliary fluids, saliva, feces, blood, pieces of tissue, etc.) and potentially from other devices or materials used in conjunction with the endoscope. Because endoscopes are used multiple times, it is important that they are completely cleaned between uses to avoid cross-contamination between devices used with them, and between different patients.

A typical cleaning regimen for cleansing of an endoscope includes wiping it down with a detergent (such as an enzymatic detergent), then soaking it in and flushing it with a same or different detergent, water, and air, and then finally drying it. The detergent provides for chemical cleaning and the flushing provides for mechanical cleaning.

Some mechanical aids are known for use in cleaning the inner channels of an endoscope, including the main working channel and other channels (e.g., accessory channels used for suction and/or passage of air or water, which collectively are referred to herein as endoscope channels or lumens). For example, several companies make linear and tapered brushes that have bristles projecting from a central shaft to provide mechanical abrasion to the lumenal surfaces in an endoscope. As another example, U.S. Pat. No. 6,699,331 to Kritzler discloses a sponge device for spreading lumenal contamination of an endoscope into a substantially uniform film on the lumenal surface so that enzymatic cleaners can more efficiently and uniformly digest the contaminating material.

However, each of these devices has some shortcomings. Because of their rigidity and shape, bristles of brush devices do not provide uniform contact with the endoscope's lumenal surfaces. The sponge device of Kritzler is not configured to provide the mechanical force/contact needed to actually remove adhering lumenal contaminants, but rather spreads them uniformly. As a result, there still exists a need for a device that overcomes these shortcomings and provides for both mechanical and chemical cleaning of endoscope lumens.

### BRIEF DESCRIPTION

In one aspect, the present disclosure provides a device for cleaning an endoscope. The device may include an elongate housing with a cavity configured to receive at least a portion of a medical device. The device may further include an inlet in fluid communication with the cavity, the inlet configured to connect to a source for providing a cleaning fluid at a pressure to the cavity through the inlet, the pressure being 100 psi or more. The device may further include an outlet in fluid communication with the cavity, the outlet configured to remove the cleaning fluid from the cavity.

In some embodiments, the pressure is 750 psi or more.

The device may include an access adapter coupled to the housing and configured to provide a seal around an outer surface of the endoscope.

A waste receptacle may be in fluid communication with the outlet.

The outlet may be configured to connect to a pump such that cleaning fluid flowing through the outlet is recirculated to the inlet.

The inlet may include a nozzle, the nozzle having a nozzle tip configured to focus the flow of the cleaning fluid on an area of the endoscope. The nozzle tip may be movable by applying a force to the inlet. The nozzle may be attached to a wall of the housing, and the wall may include an area with a flexible material configured to facilitate the movement of the nozzle tip.

In another aspect, the present disclosure provides a method for cleaning an endoscope with a cleaning device. The method may include inserting a distal end of the endoscope in a cavity of an elongate housing of the cleaning device. The method may further include placing the distal end of the endoscope adjacent to an inlet in fluid communication with the cavity. The method may further include dispensing a cleaning fluid from a nozzle of the inlet of the cleaning device, wherein the nozzle is configured to focus the flow of the cleaning fluid on an area of the endoscope, and wherein the cleaning fluid has a pressure of 100 psi or more in the inlet.

The cleaning fluid may have a pressure of at least 750 psi in the inlet.

The cleaning device may include an outlet in fluid communication with the cavity, and the method may include removing the cleaning fluid from the cavity through the outlet. The step of removing the cleaning fluid through the outlet may include moving the fluid to a waste receptacle in fluid communication with the outlet. In some embodiments, the inlet may be connected to a source and the outlet may be connected to the source, and the method may include circulating the cleaning fluid from the outlet to the inlet.

The method may include moving the nozzle to move the focus of the flow of the cleaning fluid from the area of the endoscope to a second area of the endoscope.

The method may include moving the endoscope within the cavity such that the flow of cleaning fluid from the nozzle moves from the area of the endoscope to a second area of the endoscope.

In another aspect, the present disclosure provides a device for cleaning an endoscope. The device may include an elongate housing with a cavity configured to receive at least a portion of a medical device. The device may include an inlet in fluid communication with the cavity, the inlet including a nozzle configured to focus a flow of a cleaning fluid from the inlet to the cavity on an area of the endoscope. A nozzle tip of the nozzle may be movable from a first location to a second location to change the focus of the flow of the cleaning fluid.

The inlet may be configured to dispense the cleaning fluid when the cleaning has a pressure of at least 100 psi in the inlet.

The device may include an outlet in fluid communication with the cavity, the outlet configured to remove the cleaning fluid from the cavity.

The housing of the device may include a wall with an area adjacent to the inlet, where an area of the wall having a flexible material configured to facilitate the movement of the nozzle tip.

The device may include an access adapter coupled to the housing and configured to provide a seal around an outer surface of the endoscope.

### BRIEF DESCRIPTION OF THE DRAWINGS

The embodiments of the present disclosure may be better understood with reference to the following drawings and description. The components in the figures are not necessarily to scale, with emphasis instead being placed upon illustrating the principles of the present disclosure. Moreover, in the figures, like referenced numerals designate corresponding parts throughout the different views and arrangements.

FIG. 1 shows a perspective view of an example of an endoscopic device.

FIG. 2 shows a device for cleaning an endoscope in accordance with the present disclosure.

FIG. 3 shows the device of FIG. 2 during a cleaning operation.

FIG. 4 shows the device of FIG. 2 connected to a source and a waste receptacle.

FIG. 5 shows the device of FIG. 2 in a closed-loop system.

FIGS. 6A-6C show three orientations of a nozzle for a cleaning device that is movable to focus a flow at a particular area of a medical device.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The invention is described with reference to the drawings in which like elements are referred to by like numerals. The relationship and functioning of the various elements of this invention are better understood by the following detailed description. However, the embodiments of this invention are not limited to the embodiments illustrated in the drawings. It should be understood that the drawings are not to scale, and in certain instances details have been omitted which are not necessary for an understanding of the present invention, such as conventional fabrication and assembly.

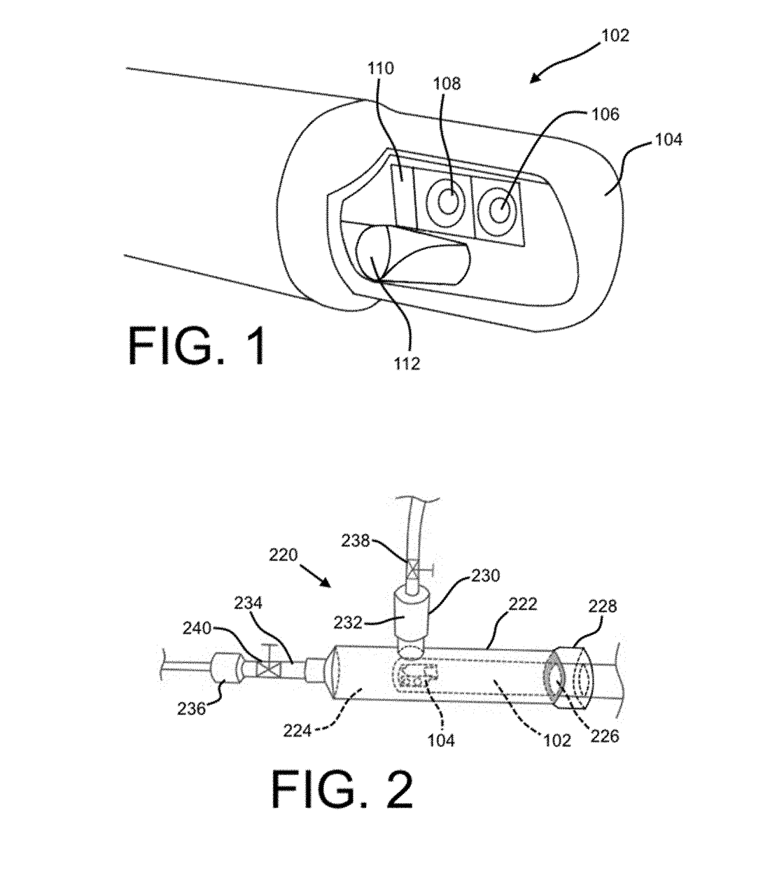

As used in the specification, the terms proximal and distal should be understood as being in the terms of a physician delivering the medical device to a patient. Hence the term “distal” means the portion of the medical device that is farthest from the physician and the term “proximal” means the portion of the medical device that is nearest to the physician. FIG. 1 shows one example of an endoscopic device, which in this case is depicted as duodenoscope (herein referred to as endoscope 102). A distal end 104 of the endoscope 102 may include several components used during medical procedures, including (but not limited to) a light 106, a camera 108, an air nozzle 110, and a movable elevator 112 that may move (e.g., open and close) to allow a physician to use a variety of tools (e.g., to perform endoscopic ultrasound). The elevator 112 may be associated with an accessory channel 113, which may extend from the distal end 104 of the endoscope 102 to a proximal end (now shown) near a handle, for example, and which may be configured to receive an accessory tool (e.g., a probe, a hemostatic clip, etc.). Due to the surface complexities of the distal end 104 of the endoscope 102, particularly surface characteristics of the elevator 112 and associated components (e.g., a wire channel receiving a wire connected to the elevator 112 and configured to move the elevator 112), endoscopic devices have proven particularly difficult to clean by existing methods. It would therefore be advantageous to provide a cleaning device that provides a precisely controlled jet of a fluid, such as a cleaning solution or other suitable agent (herein referred to as “the cleaning fluid”).

FIG. 2 shows a device 220 for cleaning the endoscope 102. The device 220 includes an elongate housing 222 with a cavity 224 configured to receive the endoscope 102. As depicted, the housing 222 includes an access opening 226, through which the distal end 104 of the endoscope 102 may be inserted. The access opening 226 may be associated with an access adapter 228, which may seal the access opening 226 once the distal end 104 of the endoscope 102 is located within the cavity 224. The access adapter 228 may be any suitable device for forming a seal at the access opening 226. For example, the access adapter 228 may be a valve that, when in a closed (sealing) state, has a compliant inner diameter that contacts an outer diameter of the endoscope 102 in a sealing manner. The adapter may be, or use components of, a Tuohy-Borst Adapter® marketed by Cook Medical Inc.® of Bloomington, Ind.

The housing 222 may include an inlet 230, as shown in FIG. 2. An inlet fitting 232 may be located adjacent to the inlet 230 and may extend externally from the housing 222. The inlet fitting 232 may be an adapter or other fitting configured to attach to plumbing equipment (described below) that facilitates flow of a cleaning fluid through the inlet 230 and into the cavity 224. The housing 222 may also include an outlet 234. The outlet 234 may be associated with an outlet fitting 236 configured to attach to plumbing equipment that facilitates the flow of the cleaning fluid out of the cavity 224.

The inlet 230 may include an inlet valve 238, and the outlet 234 may include an outlet valve 240. The inlet valve 238 may operate to control the flow rate of the cleaning fluid through the inlet 230 by at least partially restricting, and potentially completely closing, the flowpath through the inlet 230 in a controlled manner. Advantageously, by providing the inlet valve 238, the flow rate (and/or flow velocity) may be precisely controlled to adjust for desirable cleaning of different devices (e.g., different endoscope types) and/or different areas on a single endoscope 102. For example, when the endoscope 102 includes a first area that is difficult to clean but relatively durable (e.g., an area near the elevator 112 shown in FIG. 1) and also a second area that is relatively fragile (e.g., an area near the camera 108), the cleaning process may be relatively intense when cleaning the first area and relatively mild when cleaning the second area.

Similarly, the outlet 234 may include an outlet valve 240 that may operate to control the flow rate of the cleaning fluid through the outlet 234 may at least partially restricting, and potentially completely closing, the flowpath through the outlet 234 in a controlled manner. By differentiating the flow rate through the inlet 230 and the outlet 234, the amount (e.g., the volume or mass) of cleaning fluid in the cavity 224 may be adjusted. For example, when it is desired to fill (i.e., increase the amount of cleaning fluid) the cavity 224, the inlet valve 238 may be open and the outlet valve 240 may be closed (or, at least in a more restrictive state than the inlet valve 238). When it is desired to empty (i.e., decrease the amount of cleaning fluid) within the cavity 224, the inlet valve 238 may be closed or in a restrictive state and the outlet valve 240 may be open. Advantageously, by controlling the inlet valve 238 and/or the outlet valve 240, the amount of cleaning fluid within the cavity 224 may be controlled to facilitate several different types of cleaning operations (e.g., exposing the distal end 104 of the endoscope 102 with a first cleaning fluid for a relatively brief period of time without filling the cavity 224 and then soaking the distal end 104 of the endoscope in a second cleaning fluid for a longer period of time by filling the cavity 224 with the second cleaning fluid).

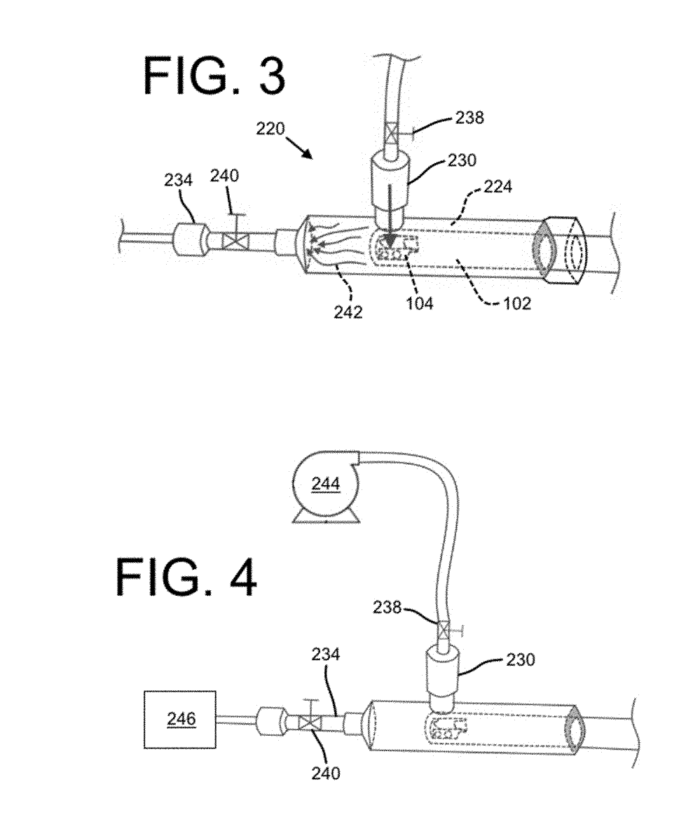

FIG. 3 shows the device 220 of FIG. 2 during a cleaning operation. As shown, a cleaning fluid 242 may flow a location upstream of the inlet 230, through the inlet 230, and into the cavity 224. In FIG. 3, the inlet valve 238 and the outlet valve 240 may be in an open state. The cleaning fluid 242 may be directed at a particular area of the distal end 104 of the endoscope 102 with relative intensity. The cleaning fluid 242 may then 1) remain in the cavity 224 such that it soaks the distal end 104 of the endoscope 102 and/or 2) it may flow towards, and into, the outlet 234. Advantageously, the cleaning fluid 242 that has been exposed to contaminants may exit the cavity 224 and replaced with new cleaning fluid 242 continuously. Advantageously, the device 220 provides the ability to continuously provide uncontaminated cleaning fluid 242 but also the advantageous cleaning properties of a fluid bath.

Referring to FIG. 4, the inlet 230 may be connected to a pump 244 or another suitable source. The pump 244 may be any suitable pump for causing a cleaning fluid to flow through the inlet 230. The pump 244 may be capable of providing the cleaning fluid with a pressure within the inlet 230, measured in pounds per square inch (psi), of about 100 psi to about 5000 psi, such as from about 750 psi to about 2000 psi. The pump 244 may be adjustable such that the intensity of the cleaning operation can be adjusted without (or, in addition to) controlling the inlet valve 238. Alternatively, the pump 244 may operate in a constant state such that the control of the cleaning fluid is provided solely by the inlet valve 238 and/or the outlet valve 240.

In the embodiment of FIG. 4, the outlet 234 leads to a waste receptacle 246, which may be any suitable receptacle for holding waste (and particularly medical waste). Advantageously, cleaning fluid may immediately enter the waste receptacle 246 without manual intervention by a medical worker. The receptacle 246 may not be required in all embodiments, particularly when it is safe to release the waste into the external environment (for example, into a sewage drain).

Alternatively, as shown in FIG. 5, the device 220 may operate in a closed environment. The outlet 234 may be associated with a flowpath 248 leading back to the pump 244, for example. This embodiment may be advantageous where a cleaning fluid may be reused through multiple cycles of a cleaning operation. For example, it is contemplated that a particular cleaning fluid may include small particles (e.g., sand particles) such that it is relatively abrasive. This particular cleaning fluid may be costly. Accordingly, it may be reused through multiple passes in a closed system. Afterwards, in a second step of the cleaning operation, a second type of cleaning fluid (e.g., a disinfectant) may be used. During this step, the device 220 may be adjusted such that it is no longer a closed loop, but rather the outlet leads to a waste receptacle (as shown in FIG. 4). Then, in a third step of the cleaning operation, the endoscope 102 may be rinsed with a third type of cleaning fluid (e.g., water).

In some embodiments, it is contemplated that a plug, a valve, and/or another suitable device may be placed within the accessory channel 113 (shown in FIG. 1) to prevent cleaning fluid from flowing proximally through the accessory channel 113 (shown in FIG. 1), particularly when the cavity 224 of the device 220 includes a cleaning fluid under pressure. In other embodiments, it may be adventurous to allow the cleaning fluid to flow proximally through the accessory channel 113, which may clean and/or disinfect the accessory channel 113. Optionally, the proximal end of the accessory channel may be connected to the pump 244 (or other source) via tubing so that cleaning fluid flowing through the accessory channel 113 may be re-circulated or directed to a waste receptacle. Additionally or alternatively, it is contemplated that the proximal end of the accessory channel 113 may be placed in a suitable container or other device configured to capture cleaning fluid through and out of the proximal end of the accessory channel 113, for example.

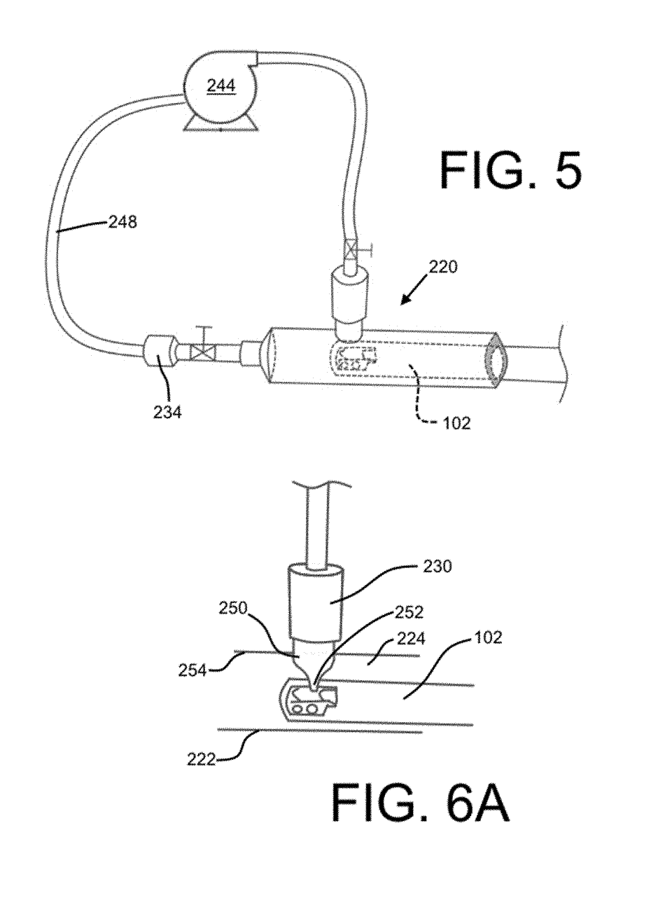

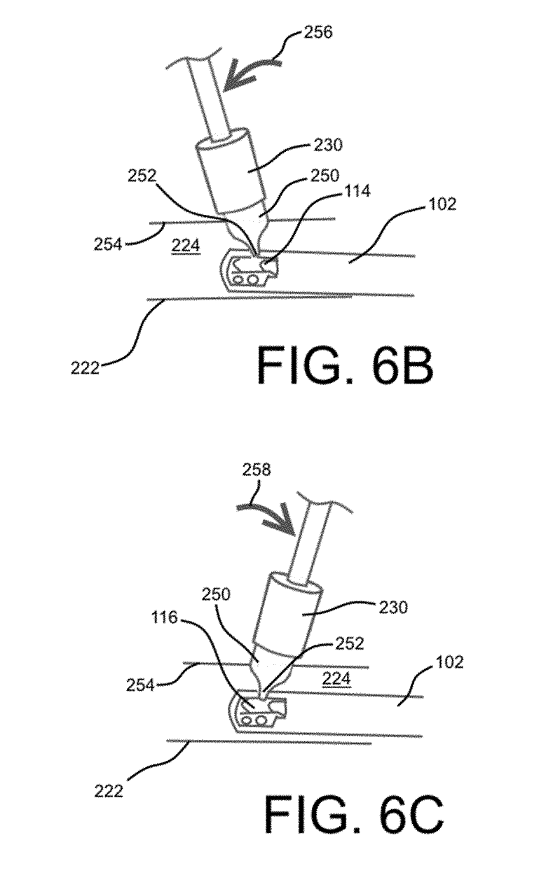

As depicted in FIG. 6A, in some embodiments, the inlet 230 may include a nozzle 250. The nozzle 250 to dispense the cleaning fluid, and may be configured to direct the flow of a cleaning fluid in a particular direction such that it is precisely focused on a particular area of the endoscope 102, for example. The nozzle 250 may extend inward from an interior wall of the cavity 224 such that the tip 252 of the nozzle 250 is located in close proximity to a particular area of the endoscope 102. It is contemplated that the tip 252 of the nozzle 250 may extend at least partially into a channel or other cavity of the endoscope 102, for example (e.g., at a location associated with an elevator of a duodenoscope).

In some embodiments, the nozzle 250 may be movable. For example, at least a portion of the wall 254 of the housing 222 adjacent to the nozzle 250 may be formed of a relatively compliant material (such as, for example, a rubber) such that the aim of the nozzle 250 can be adjusted by a user, such as applying a force in a particular direction on the inlet 230 by hand (or by automatic means) to pivot the tip 252 of the nozzle 250.

FIG. 6B shows the nozzle 250 in a second orientation such that the tip 252 of the nozzle 250 is aimed at a first area 114 of the endoscope 102. It is contemplated that the user may adjust the flow rate of the cleaning fluid flowing through the nozzle 250 for optimal cleaning of the first area 114, though adjusting the flow rate is not necessary and/or desirable in all contemplated cleaning operations. The nozzle 250 may be moved from the orientation of FIG. 6A to the orientation of FIG. 6B by applying a force on the inlet 230 in the direction associated with the arrow 256 of FIG. 6B, for example. While not shown, it is contemplated that the nozzle 250 may be moved automatically by a device such as an electric motor, a pneumatic device, or the like. Additionally or alternatively, while not shown, the nozzle 250 may include a locking device to lock the nozzle 250 in a particular position with respect to the housing 222.

Similarly, the nozzle 250 may be movable to a third orientation depicted in FIG. 6C such that the flow through the nozzle 250 is focused on a second area 116 of the endoscope 102. The nozzle 250 may be moved to this orientation from another orientation (such as the orientation of one of FIG. 6A and FIG. 6B) by applying a force on the inlet 230 in the direction associated with the arrow 258 of FIG. 6A. The nozzle 250 may be moved repeatedly back-and-forth (front to back, side to side, etc.) during a cleaning operation. While not shown, it is also contemplated that the nozzle 250 may be movable vertically (with respect to the views of FIG. 6A, FIG. 6B, and FIG. 6C) to further control the proximity of the tip 252 of the nozzle 250 with respect to the endoscope 102. The flow may be automatically or manually adjusted to correspond with the position of the nozzle 250 (i.e., the flow may be increased when the nozzle 250 directs flow towards certain hard-to-reach cavities of the endoscope 102, and/or may be decreased when the nozzle 250 is oriented to direct flow at fragile portions of the endoscope 102). The nozzle 250 may be moved in only one direction (e.g., it may be pivotably about only one axis), or it may be movable in many directions. It is contemplated that the nozzle 250 may be rotated 360°, may be moved linearly, and/or may be moved vertically with respect to the housing 222. Advantageously, the nozzle 250 may be therefore be configured such that it is adjustable to focus high-pressure flow precisely on particular areas of the endoscope 102 or another medical device to ensure suitable mechanical and chemical cleaning.

In addition to, or as an alternative to, moving the nozzle 250, it is contemplated that the endoscope 102 may be moved within the cavity such that the focus of the flow out of the nozzle tip 252 moves with respect to the endoscope 102. This movement may include moving the endoscope along its longitudinal axis, vertically to adjust the proximity to the nozzle tip 252, and/or by rotating the endoscope 102. The housing 222 may be formed of a transparent material such that the position of the endoscope 102 is visible to a user. It is contemplated that the housing may include visual indicators to provide a reference-point to the user when adjusting the position of the endoscope 102 and/or when initially placing the endoscope 102 into the cavity prior to initiation of cleaning.

The figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

We claim:

1. A device for cleaning an endoscope, the device comprising:
an elongate housing with a cavity configured to receive at least a portion of a medical device;
an inlet in fluid communication with the cavity, the inlet configured to connect to a source for providing a cleaning fluid at a pressure to the cavity through the inlet, the pressure being 100 psi or more; and
an outlet in fluid communication with the cavity, the outlet configured to remove the cleaning fluid from the cavity.

2. The device of claim 1, wherein the pressure is 750 psi or more.

3. The device of claim 1, further comprising an access adapter coupled to the housing and configured to provide a seal around an outer surface of the endoscope.

4. The device of claim 1, further comprising a waste receptacle in fluid communication with the outlet.

5. The device of claim 1, wherein the outlet is configured to connect to a pump such that cleaning fluid flowing through the outlet is recirculated to the inlet.

6. The device of claim 1, wherein the inlet includes a nozzle, the nozzle having a nozzle tip configured to focus flow of the cleaning fluid on an area of the endoscope.

7. The device of claim 6, wherein the nozzle tip is movable by applying a force to the inlet.

8. The device of claim 7, wherein the nozzle is attached to a wall of the housing, and wherein the wall includes an area with a flexible material configured to facilitate the movement of the nozzle tip.

9. A method for cleaning an endoscope with a cleaning device, the method comprising:
inserting a distal end of the endoscope in a cavity of an elongate housing of the cleaning device;
placing the distal end of the endoscope adjacent to an inlet in fluid communication with the cavity; and
dispensing a cleaning fluid from a nozzle of the inlet of the cleaning device, wherein the nozzle is configured to focus flow of the cleaning fluid on an area of the endoscope, and wherein the cleaning fluid has a pressure of 100 psi or more in the inlet.

10. The method of claim 9, wherein the cleaning fluid has a pressure of at least 750 psi in the inlet.

11. The method of claim 9, wherein the cleaning device includes an outlet in fluid communication with the cavity, and wherein the method comprises removing the cleaning fluid from the cavity through the outlet.

12. The method of claim 11, wherein the step of removing the cleaning fluid through the outlet includes moving the fluid to a waste receptacle in fluid communication with the outlet.

13. The method of claim 11, wherein the inlet connected to a pump and wherein the outlet is connected to the pump, the method further comprising circulating the cleaning fluid from the outlet to the inlet.

14. The method of claim 9, further comprising moving the nozzle to move the focus of flow of the cleaning fluid from the area of the endoscope to a second area of the endoscope.

15. The method of claim 9, further comprising moving the endoscope within the cavity such that the flow of cleaning fluid from the nozzle moves from the area of the endoscope to a second area of the endoscope.

16. A device for cleaning an endoscope, the device comprising:
an elongate housing with a cavity configured to receive at least a portion of a medical device; and
an inlet in fluid communication with the cavity, the inlet including a nozzle configured to focus a flow of a cleaning fluid from the inlet to the cavity on an area of the endoscope,
wherein a nozzle tip of the nozzle is movable from a first location to a second location to change the focus of the flow of the cleaning fluid.

17. The device of claim 16, wherein the inlet is configured to dispense the cleaning fluid when the cleaning has a pressure of at least 100 psi in the inlet.

18. The device of claim 16, further comprising an outlet in fluid communication with the cavity, the outlet configured to remove the cleaning fluid from the cavity.

19. The device of claim 16, where the housing includes a wall with an area adjacent to the inlet, an area of the wall having a flexible material configured to facilitate movement of the nozzle tip.

20. The device of claim 16, further comprising an access adapter coupled to the housing and configured to provide a seal around an outer surface of the endoscope.
